(19) United States
(12) Patent Application Publication
(10) Pub. No.: US 2008/0089466 A1
(43) Pub. Date: Apr. 17, 2008

(54) X-ray CT examination installation and CT methods of examining objects

(75) Inventors: Martin Munker, Gevelsberg (DE); Faisal Laakel, Bochum (DE); Matthias Helliing, Herne (DE)

Correspondence Address: GRAYBEAL, JACKSON, HALEY LLP, 155 - 108TH AVENUE NE, SUITE 350, BELLEVUE, WA, 98004-5973, US

(73) Assignee: YXLON International X-Ray GmbH, Hamburg (DE)

(21) Appl. No.: 11/899,983

(22) Filed: Sep. 6, 2007

(30) Foreign Application Priority Data
Sep. 6, 2006 (DE) 102006041850.6

(51) Int. Cl. A61B 6/02

(52) U.S. Cl. 378/14; 378/17

(57) ABSTRACT

The invention relates to an X-ray CT examination installation, with an X-ray tube with a focus, which creates a fan beam or a conical beam which X-rays the whole of a detector located at a defined distance from the focus, and with an examination carriage, for receiving an object to be examined, which has an axis of rotation rotatable perpendicular to the plane of the fan beam or to the central axis of the conical beam. It is provided according to the invention that the examination carriage can be fixed on at least one measuring point, wherein each of these measuring points is arranged such that the axis of rotation is on a measuring line which emanates from the focus and includes a tilt angle ∝ with the central axis of the fan beam or the conical beam. The invention also relates to a CT method of examining objects, in particular of different sizes, by means of an X-ray CT examination installation according to the invention.

Priority to German patent application number DE 10 2006 041 850.6 filed on 6 Sep. 2006 under 35 U.S.C. §119(a) is claimed, said application being incorporated herein by reference.

### FIELD OF THE INVENTION

The invention relates to an X-ray CT examination installation, with an X-ray tube with a focus, which creates a fan beam or a conical beam which X-rays the whole of a detector located at a defined distance from the focus, and with an examination carriage, for receiving an object to be examined, which has an axis of rotation rotatable perpendicular to the plane of the fan beam or to the central axis of the conical beam. The invention also relates to a CT method of examining objects, in particular of different sizes, by means of an above-named X-ray CT examination installation.

### BACKGROUND

There are currently two examination methods in industrial computed tomography (CT). One is a translation/rotation tomography (2^{nd} generation) and the other a fan beam tomography (3^{rd} generation). In both cases a fan beam which X-rays the whole of a one-dimensional detector is masked before a focal point, the focus, of an X-ray source. Both the X-ray source and also the detector are fixed. An object to be examined, which is rotated about an axis perpendicular to the plane of the fan beam, is inserted between them into the fan beam in order that the object can be reconstructed. The distance between X-ray tube and detector can be altered, likewise the position of the object which is arranged on a turntable so that the geometric magnification can be matched to the requirements in each case. The individual horizontal layers of the object are recorded by progressively changing the height of the object or the X-ray tube and the detector. Instead of using a fan beam it is also possible to use a conical beam and to project this onto a two-dimensional detector. A layer-by-layer scanning can then be dispensed with, depending on the size of the object.

In fan beam tomography a complete measured data record is created because the whole of the object to be examined lies in the section plane in the fan beam and projections are recorded from at least 180° plus aperture angle of the fan beam. This method is fast, but the size of the beam fan determines the maximum size of the object which can be tomographed in this arrangement. This size is also called measuring circle. The relationship also applies in reverse, i.e. in order that a larger object can be tomographed, the measuring circle must be larger, i.e. a fan beam with larger aperture angle and thus also a larger detector are used. With this method even comparatively small objects require large installations with a long detector and a large distance between focus and detector. Technical limitations result from the maximum angle of radiation of the ray source which limits the aperture angle, and the size of the detector.

If the fan beam does not cover the whole object, this fan beam can be artificially widened by moving either the ray source and detector or the object sideways. This is translation/rotation tomography. However, there must be alternating linear and rotary movements here, which is time-consuming and also requires for the transverse movement a linear axis which, over the whole distance covered, must ensure with a high degree of accuracy the right-angled alignment of the axis of rotation to the plane of the fan beam.

If, instead of a two-dimensional fan beam, a three-dimensional conical beam is used, the available examination volume is confined between the focus of the X-ray source and the corners of the mostly square two-dimensional surface detector or the edge contour of an inserted image-recording device. Otherwise the above statements apply by analogy.

Given a typical size and quantity distribution of objects to be examined—there are frequently many small and few large objects—it has hitherto been necessary to design an examination installation such that the large objects can be examined in all cases. Disadvantages result from this with regard to the size of the whole examination installation and with regard to the achievable measuring time.

### SUMMARY OF THE INVENTION

The object of the invention is to provide a compact and rapid X-ray CT examination installation which has been mechanically simplified compared with the conventional design, for the examination of objects of clearly differing sizes. For this, a method with which such an X-ray CT installation is operated is also to be provided.

The object is achieved by an X-ray CT examination installation with the features of claim 1. Because the examination carriage can be fixed at measuring points which are on a measuring line, the device has a simple mechanical design. By aligning the measuring line on a straight line which passes through the focus and has a tilt angle vis-à-vis the central axis of the X-ray, objects of a predetermined size to be examined can be examined by a smaller installation than is the case with an installation according to the state of the art of translation/rotation tomography and fan beam tomography. This is because the distance between the measuring line and one of the marginal rays of the fan beam is always greater than the distance from the central axis to the respective marginal ray. It is possible according to the invention for there to be only one single measuring point. A very simple mechanical design of the design is thereby obtained. However, it is also equally possible according to the invention for there to be a whole row of measuring points at which the examination carriage can be fixed. A flexible examination arrangement is thereby created with which objects of different sizes can also be examined easily and quickly in a very simple manner.

The tilt angle between the central axis of the fan beam or the conical beam and the measuring line is preferably between 5° and 40°. There is thus a clear expansion of the size of objects to be measured for the same overall height; alternatively, the overall height of the X-ray CT examination installation can be reduced for a predetermined object size. The tilt angle is particularly preferably 15°.

A particularly preferred embodiment of the invention provides that the measuring line stands perpendicular to the surface of the detector. With such a design it is not necessary to carry out a geometric adjustment to the acquired measuring data, as no distortion results from the geometry of the installation. Moreover, such an arrangement is very simple to realize.

A further advantageous development of the invention provides that the detector is connected to a drive unit and is displaceable perpendicular to the measuring line within the plane of the fan beam or within the plane of the detector in the case of a conical beam. It is thereby possible with simple mechanical outlay to create a virtual detector which is larger than the actually existing detector. The central axis preferably meets the synthesized—virtual—detector centrally. Unlike the conventional measuring circle extension which synthesizes a fixed predetermined detector size from several, fixed positions, the free displacement of the detector enables its overall length and thus all of the measured data to be used here loss-free.

Another preferred development of the invention provides that the central axis of the fan beam or the conical beam, but not the measuring line, stands perpendicular to the surface of the detector. The advantage—which has already been described above—thereby also results that either larger objects can be examined for a predetermined size of the installation or the size can be reduced for a predetermined size of an object as the distance from the measuring point to one of the marginal rays is greater than from the central axis. In this case, although a maximum line length is guaranteed with full use of the radiation angle of the X-ray tube, a geometric adjustment of the acquired data must be accepted.

A further advantageous development of the invention provides that the examination carriage can be continuously fixed at measuring points on the measuring line. It is thereby possible to continuously vary the magnification. In addition, a large bandwidth of object sizes is also permitted, all of which can be properly examined.

A further advantageous development of the invention provides that the measuring line is embodied by a guide rail on which the examination carriage can be moved. A very simple mechanical design is thereby obtained which simultaneously allows a very precise positioning of the examination carriage and thus of the axis of rotation. It is possible hereby firstly that the examination carriage can be fixed continuously—i.e. at every point of the examining line—at a row of specific points—i.e. to examine objects of a predetermined size—or else at a single point.

A further advantageous development of the invention provides that at every measuring point there is free rotation of the examination carriage with an object to be examined secured thereon. In every case, all data necessary for the CT method are obtained when examining the object.

A further advantageous development of the invention provides that in every measuring point during the rotation of the object the latter always remains within a marginal ray which emanates from the focus and meets the detector at its edge. It is thereby likewise guaranteed that all the object data necessary for the examination can be acquired without a displacement of the detector or a movement of the axis of rotation perpendicular to the measuring line being necessary.

Additionally the object is also achieved by a CT method with the features of claim 11. Because the axis of rotation of the examination carriage is spatially fixed close to the focus for positioning within the fan beam, wherein during rotation about the axis of rotation the object never projects beyond both marginal rays, the advantages already named above in relation to the X-ray CT installation according to the invention are achieved: namely the possibility of being able to use smaller detectors and consequently also a smaller-sized X-ray CT examination installation for the examination of objects of predetermined size. This leads to a cost saving vis-à-vis the known X-ray CT methods. At least, the favourable part-fan procedure can—if the whole of the object is not already lying in the beam window anyway—also always be used with the cited advantages. Furthermore the axis of rotation need stand as precisely perpendicular as possible to the plane of the fan beam only at the point at which it has been positioned to examine the object. By part-fan procedure is meant in the context of this application a method which, unlike half-detector methods in which a complete information half is generated from the data of the other side, artificially produces specific areas only, while the areas in the middle are, looked at in this way, measured “overlapping”: a more favourable signal-to-noise ratio can be achieved via calculation of the data. This is of interest if there is a very precise evaluation only in the centre of the image—which need not necessarily be the centre of the part.

Additionally the object is also achieved by a CT method with the features of claim 12. Because the positioning of the object depends on its size, small first objects can be positioned such that they are wholly pierced by the fan beam, which leads to a very fast examination of these small first objects. Additionally, larger second objects which are not wholly pierced by the fan beam can be positioned close to the detector and a fan beam tomography carried out. This is also a significantly faster method than the translation/rotation tomography. In addition, objects of medium size can either be examined at low magnification (i.e. close to the detector) in the full beam and thus more quickly, or else somewhat slower but at higher magnification in the part- or half-beam method. According to the invention it need only be guaranteed that in the case of the larger second objects the axis of rotation is positioned such that the measuring circle never projects beyond the two marginal rays.

An advantageous development of the invention provides that a first object is arranged for examination so remote from the focus on the measuring line that it just fails to project beyond the fan beam or cone envelope beam throughout the examination. It is thereby possible to tomograph a small first object of maximum size using fan beam tomography.

A further advantageous development of the invention provides that a second object is arranged for examination so remote from the focus on the measuring line that it just fails to project beyond the two marginal rays throughout the examination. It is thereby possible that as large as possible a second object can be tomographed by means of fan beam tomography.

A further advantageous development of the invention provides that during the measurement the detector is moved by means of the drive unit from its one extreme position into its other extreme position or its other extreme positions. As already stated above with regard to the device, a synthesis of a larger detector than the actually existing detector is thereby achieved. As a result, either larger objects to be examined can be examined for a constant size of the device or the size can be reduced for a predetermined size of the object.

Further advantages and details of the invention are described with the help of the embodiment examples, described below, represented in the Figures. There are shown in:

### BRIEF DESCRIPTION OF THE DRAWINGS

FIG. 1 a schematic top view of a first embodiment example of an X-ray CT examination installation according to the invention in which the examining line stands perpendicular to the surface of the detector, and

FIG. 2 a schematic top view of a second embodiment example of an X-ray CT examination installation according to the invention in which the central axis of the X-ray beam stands perpendicular to the surface of the detector.

### DETAILED DESCRIPTION OF THE INVENTION EMBODIMENTS

It is assumed below in respect of the two embodiment examples that a fan beam 3 is present. However, the statements also apply analogously to a conical beam, which is immediately clear to a person skilled in the art as he knows the different geometries of the installations in principle and can transfer the embodiment according to the invention with fan beam 3 to the conical beam without difficulty.

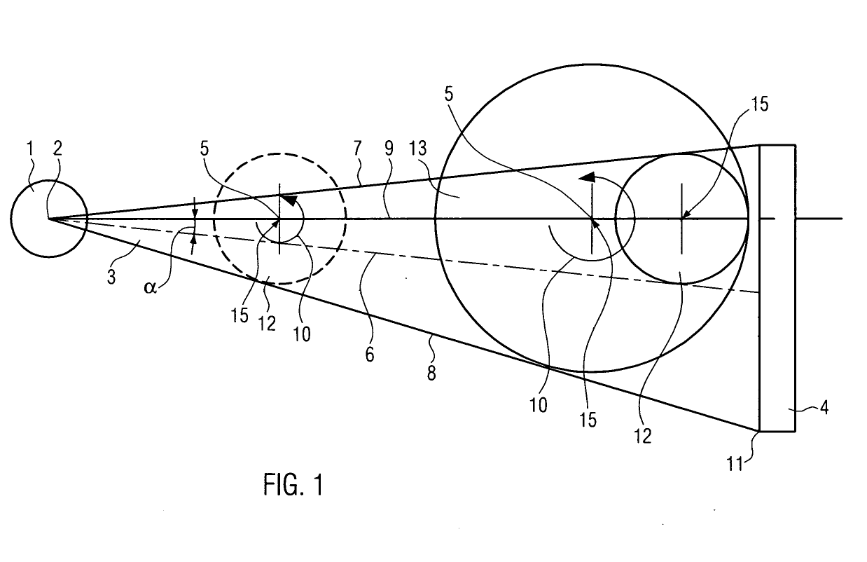

A top view of an X-ray CT examination installation is schematically represented in FIG. 1. An X-ray tube 1 has a focus 2 from which an X-ray beam in the form of a fan beam 3 emanates. This fan beam 3 meets a detector 4 the whole of which is X-rayed by the fan beam 3. Such an arrangement is well known from industrial CT. The distance between detector 4 and focus 2 can be varied in order to achieve as good as possible an image geometry for an object to be examined. As only a thin slice in the plane of the fan beam 3 of the object to be examined can be tomographed by the fan beam 3, both the X-ray tube 1 and the detector 4 can be moved vertically. The object to be examined is X-rayed layer-by-layer. Alternatively it is also possible to vary the height of the object or to combine both methods.

A carriage (not shown) on which an object to be examined can be fixed is arranged between the focus 2 and the detector 4. The examination carriage rotates about an axis of rotation 5 which stands perpendicular to the plane of the fan beam 3. Data records of the object are thus generated along various irradiation paths. The object to be examined is then reconstructed from these data records. This is a nondestructive analysis of defects in the object to be examined, for example faults in a casting, or the ascertaining of geometric properties.

Objects of different sizes have different-sized measuring circles 12, 13. A measuring circle 12, 13 is determined on the one hand by the shape of the object and the position of the axis of rotation 5 about which the object is rotated during examination. The radius of the measuring circle 12, 13 corresponds to the greatest distance of any point of the object from the axis of rotation 5. In the case of “rotation-symmetrical” wheels this corresponds to the radius of the wheel if it is attached to the examination carriage such that its centre coincides with the axis of rotation 5.

Previously it was possible to examine by means of a fan beam tomography method objects whose measuring circle 12 lay wholly in the fan beam 3. This greatly limited the largest possible objects, as the latter were recordable with only a very small degree of magnification, as they had to be arranged in the immediate vicinity of the detector 4. On the other hand it was possible, with smaller objects with a smaller measuring circle 12, to also position these along a central axis 6 inside the fan beam 3 at a greater distance from the detector 4.

In order to tomograph objects with larger measuring circles 13, it was previously necessary to use the above-described translation/rotation tomography, which was time-consuming and technically more expensive to realize. Another possibility for such large objects was to use a part-fan procedure.

The invention now opens up the possibility of carrying out both a fan beam tomography and a part-fan procedure in the same X-ray CT examination installation. It is thereby possible to fix the suitable method individually in each case according to the size of the object and thus its measuring circle 12, 13.

In order to achieve as fast a measurement as possible, a fan beam tomography is regularly carried out for small objects with a first measuring circle 12 which lies wholly in the fan beam 3. However, a part-fan procedure can also be carried out with a first measuring circle 12 in the case of such larger objects if the geometric imaging conditions can then be improved in the respective application. The result is that with a part-fan procedure an object with a predetermined measuring circle 12, 13 can be arranged closer to the focus 2 and yet a reliable X-ray is still achieved.

By tilting the measuring line 9 by the tilt angle α vis-à-vis the central axis 6 the measuring line 9—or its virtual extension—does not meet the detector 4 centrally. This means that a fan beam tomography can only be carried out for smaller objects than if the examining line 9 were to run along the central axis 6. This becomes clear from FIG. 1 because two measuring points 15 for objects with a first measuring circle 12 of the same size are shown. In the case of the left-hand measuring point 15, the axis of rotation 5 lies so close to the focus 2 that the first measuring circle 12 does not lie wholly in the fan beam 3. Thus, at this measuring point 15, only a part-fan procedure is possible by rotation 10 of the object about the axis of rotation 5. On the other hand, the latter is wholly covered by the fan beam 3 at the right-hand measuring point 15 for the same measuring circle 12. Thus a fan beam tomography can be carried out here. This is faster, but the magnification in the left-hand measuring point 15 is greater, with the result that the method which is necessary in each case can be chosen.

For an object with a larger, second measuring circle 13, the part-fan procedure is used. The measuring point 15 is chosen such that during a rotation 10 about the axis of rotation 5 it just reaches the surface of the detector 4. The largest possible second measuring circle 13 is thereby fully utilized.

For every measuring circle 12, 13, in addition to the necessary free rotatability vis-à-vis the detector 4—and thus a sufficient distance of the measuring point 15 from same—it is also necessary that it does not project over both marginal rays 7, 8. Otherwise the part-fan procedure cannot be used, but a displacement of the measuring point 15 perpendicular to the measuring line 9—according to the translation/rotation tomography—would have to be carried out. However, this would result in considerable additional mechanical outlay for the device.

The device according to the invention according to FIG. 1 is thus very well suited to examine differently sized objects—which can actually vary quite markedly in size—in one and the same device. A linear movement is not necessary during the measuring procedure for any of the differently sized objects, rather merely the rotation 10 about the axis of rotation 5. Considerable time is thereby saved.

A simplification of the mechanism is achieved because the object is recorded in each case at measuring points 15 on the measuring line 9 and the carriage moves with merely one degree of freedom. No linear scanning axis is thus required. The imaging conditions can be optimized in respect of the resolution via the position of the axis of rotation 5 on the measuring line 9. Thus by means of the part-fan procedure it is possible in particular to tomograph an object of average size both at low magnification—as is shown in the right-hand position of the first measuring circle 12—and at high magnification—in the first measuring circle 12 shown on the left.

If the installation is always to examine identical predetermined objects, their size and frequency distribution can be used to optimize the length of the detector 4 and its lateral displacement—which follows from the tilting by the tilt angle α to the central axis 6—in respect of the field size and measuring time. No geometric adjustment of the line profile is necessary, because of the perpendicular alignment of the measuring line 9 to the detector 4.

The device according to the invention can also be designed in a “slimmed-down version” such that merely one fixing of the axis of rotation 5 at discrete measuring points 15 on the virtually present measuring line 9 is possible. A movement of the examination carriage along this virtual measuring line 9 is then not possible. An even simpler mechanism and thus lower costs are thereby achieved.

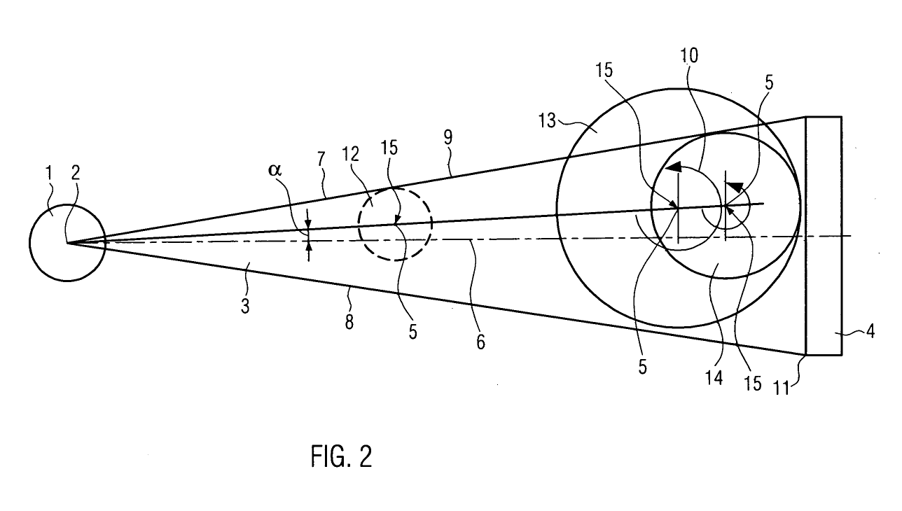

The further embodiment represented in FIG. 2 of an X-ray CT examination installation according to the invention is in principle structurally very similar to that of FIG. 1. For this reason, only the differences compared with the first embodiment example of FIG. 1 are described in more detail. Parts which are identical or have the same effect are given the same reference numbers.

The main difference between the first embodiment example of FIG. 1 and the second embodiment example of FIG. 2 is that the central axis 6 of the fan beam 3 stands perpendicular to the surface of the detector 4 and no longer the measuring line 9. As the measuring line 9 tilts here, also, by the tilt angle α vis-à-vis the central axis 6 an angle-dependent correction of the line profile for the apparently inclined detector 4 is obtained with the object data acquired during the examination.

Any desired tilt angle α can in principle be chosen, wherein the optimum tilt angle α is calculated from the size and frequency distribution of the objects to the examined in conjunction with the size of the detector and the distance between focus 2 and detector 4. The field size and measuring time can be optimized by the thus-chosen tilt angle α.

Three different measuring points 15 are given in all in the embodiment example of FIG. 2, wherein in the case of the left-hand measuring point 15 there is a very small first measuring circle 12 which lies wholly in the fan beam 3, with the result that a fan beam tomography can be carried out. The second measuring circle 13 is significantly larger than the first measuring circle 12 and cannot be positioned at any point on the measuring line 9 which makes possible a full covering of the fan beam 3. It is therefore brought as close as possible to the detector 4 and a part-fan tomography must be carried out here. The third measuring point 15 with a medium-sized third measuring circle 14 is just large enough that—given a sufficient distance from the detector 4—to lie inside both the first marginal ray 7 and the second marginal ray 8. A fan beam tomography can thereby be carried out for it (as for the first measuring circle 12).

With this device also, merely a continuous movement with a degree of freedom takes place along the measuring line 9; in the case of discrete measuring points 15 only a fixing to same is possible. No linear scanning axis is necessary and this results in a simplification of the mechanism. With this device also, an optimizing of the imaging conditions is possible in respect of the resolution—as described above—with the result that a small object can be tomographed both at low magnification in the full fan beam 3 and at high magnification with the part-fan procedure.

Overall, with an embodiment according to FIG. 2, an optimized geometry is obtained with a maximum line length accompanied by full utilization of the radiation angle of the X-ray tube 1, wherein however a geometric correction is necessary.

Ultimately, the following advantages are achieved by both embodiment examples: a reduction in the length of the detector 4 relative to the largest achievable measuring circle 12, 13, 14 is possible, the costs of the device thereby being reduced. In addition, both the distance between focus 2 and detector 4 can be reduced—which leads to a shortening of the measuring time—and a reduction in the size of the installation achieved—which leads to a reduction in the costs for the installation. Due to the smaller size, the protective case can also be reduced in size, again with a saving on costs.

### LIST OF REFERENCES

1 X-ray tube
2 focus
3 fan beam
4 detector
5 axis of rotation
6 central axis
7 first marginal ray
8 second marginal ray
9 measuring line
10 rotation
11 edge of the detector
12 first measuring circle
13 second measuring circle
14 third measuring circle
15 measuring point
∝ tilt angle

What is claimed:

1. X-ray CT examination installation, comprising an X-ray tube having a focus, which creates a fan beam or a conical beam of X-ray radiation defining a plane or a central axis, respectively, and which X-rays the whole of a detector located at a defined distance from the focus, and an examination carriage, for receiving an object to be examined, which has an axis of rotation rotatable perpendicular to the plane of the fan beam or the central axis of the conical beam,
characterized in that
the examination carriage can be fixed at least one measuring point, wherein the at least one measuring points is arranged such that the axis of rotation is located on a measuring line that emanates from the focus and includes a tilt angle (α) with the central axis of the fan beam or the conical beam.

2. X-ray CT examination installation according to claim 1, characterized in that the tilt angle (∝) is between 5° and 40°, in particular 15°.

3. X-ray CT examination installation according to claim 1, characterized in that the measuring line stands perpendicular to the surface of the detector.

4. X-ray CT examination installation according to claim 3, characterized in that the detector is connected to a drive unit and can be moved perpendicular to the measuring line within the plane of the fan beam or within a plane of the detector in the case of a conical beam.

5. X-ray CT examination installation according to claim 4, characterized in that the central axis meets a synthesized detector, which results from the extreme positions on the basis of its movement, centrally.

6. X-ray CT examination installation according to claim 1, characterized in that a central axis of the fan beam or the central axis of the conical beam stands perpendicular to the surface of the detector.

7. X-ray CT examination installation according to claim 1, characterized in that the examination carriage can be fixed continuously at measuring points on the measuring line.

8. X-ray CT examination installation according to claim 1, characterized in that the measuring line is embodied in a guide rail on which the examination carriage can be moved.

9. X-ray CT examination installation according to claim 7, characterized in that in every measuring point there is a free rotation of the examination carriage with the object to be examined fixed thereon.

10. X-ray CT examination installation according to claim 9, characterized in that in every measuring point during the rotation of the object the latter always remains within a marginal ray which emanates from the focus and meets the detector at an edge thereof.

11. (canceled)

12. (canceled)

13. (canceled)

14. (canceled)

15. (canceled)
